(19) United States
(12) Patent Application Publication
(10) Pub. No.: US 2019/0290142 A1
(43) Pub. Date: Sep. 26, 2019

(54) PULSE WAVE MEASUREMENT DEVICE, PULSE WAVE MEASUREMENT METHOD, AND BLOOD PRESSURE MEASUREMENT DEVICE

(71) Applicants: OMRON CORPORATION, Kyoto-shi (JP); OMRON HEALTHCARE CO., LTD., Muko-shi (JP)

(72) Inventors: Daisuke ISHIHARA, Kyoto (JP); Yasuhiro KAWABATA, Kyoto (JP)

(73) Assignees: OMRON CORPORATION, Kyoto-shi (JP); OMRON HEALTHCARE CO., LTD., Muko-shi (JP)

(21) Appl. No.: 16/441,084

(22) Filed: Jun. 14, 2019

Related U.S. Application Data
(63) Continuation of application No. PCT/JP2017/038870, filed on Oct. 27, 2017.

(30) Foreign Application Priority Data
Dec. 28, 2016 (JP) 2016-254771

(51) Int. Cl. A61B 5/022; A61B 5/021; A61B 5/0225; A61B 5/00

(52) U.S. Cl. CPC A61B 5/02225; A61B 5/02125; A61B 5/11; A61B 5/6831; A61B 5/0225

(57) ABSTRACT

A first and second pulse wave sensor is mounted on the belt in a state of being separated from each other in the width direction. The belt is mounted around the measurement site, the member presses the first and second pulse wave sensors against the measurement site with a predetermined pressing force. Each of the pulse wave sensors detects a pulse wave in a facing portion of an artery passing through the measurement site. The body motion detection unit detects the presence or absence of body motion. When there is no body motion, the control unit sets a force of the pressing member to a pressing force to measure a pulse wave with the sensors. When there is body motion, the control unit sets a force of the pressing member to a second force lower than the first force and higher than zero and interrupts measurement of a pulse wave.

### CROSS-REFERENCE TO RELATED APPLICATION

This is a continuation application of International Application No. PCT/JP2017/038870, with an International filing date of Oct. 27, 2017, which claims priority of Japanese Patent Application No. 2016-254771 filed on Dec. 28, 2016, the entire content of which is hereby incorporated by reference.

### TECHNICAL FIELD

The present invention relates to a pulse wave measurement device and a pulse wave measurement method, and more specifically, relates to a pulse wave measurement device and pulse wave measurement method for non-invasively measuring the transit time of pulse waves propagating in an artery (pulse transit time; PTT).

In addition, the present invention relates to a blood pressure measurement device that includes this pulse wave measurement device and calculates the blood pressure by using a correspondence equation between pulse transit time and blood pressure.

### BACKGROUND ART

Conventionally, for example, as disclosed in Patent Literature 1 (JP H02-213324 A), there is known a technique for fixedly arranging a small cuff 13 and a middle cuff 12 in a cuff 10 in a state of being separated from each other in the width direction of the cuff 10 (corresponding to the longitudinal direction of the upper arm) to measure a time difference between the respective pulse wave signals detected by the small cuff 13 and the middle cuff 12 (pulse transit time). In the cuff 10, a large cuff 11 for blood pressure measurement by oscillometric method is arranged along the space between the small cuff 13 and the middle cuff 12.

### SUMMARY OF INVENTION

When continuous measurements of pulse wave or blood pressure over a fixed period are to be obtained, since it is necessary to keep pressing the measurement site of the subject, the subject is physically burdened.

On the other hand, when the subject of the pulse wave is not in a resting state, there may be a case where a component resulting from the body motion of the subject is superimposed on the pulse wave signal, and the pulse transit time cannot be accurately measured. Therefore, when there is a body motion of the subject (when the pulse transit time cannot be measured), for example, it is conceivable to stop the pressing at the measurement site to relieve the physical burden of the subject. Thus, relieving the physical burden as much as possible when the pulse wave or the blood pressure is measured improves the convenience of the subject.

Thus, an object of the present invention is to provide a pulse wave measurement device and a pulse wave measurement method for controlling pressing force on a measurement site with a novel control method so as to improve the convenience of the subject in consideration of the body motion of the subject.

In addition, an object of the present invention is to provide a blood pressure measurement device that includes this pulse wave measurement device and calculates the blood pressure by using a correspondence equation between pulse transit time and blood pressure.

In order to solve the above-mentioned problem, a pulse wave measurement device of the present disclosure comprises:

a belt to be mounted around a measurement site of a subject;

at least one pulse wave sensor mounted on the belt, the at least one pulse wave sensor configured to detect a pulse wave of an artery passing through the measurement site;

a pressing member mounted on the belt, the pressing member configured to vary a pressing force to press the at least one pulse wave sensor against the measurement site;

a body motion detection unit configured to detect presence or absence of body motion of the subject; and

a control unit configured to set a pressing force of the pressing member to a first pressing force when there is no body motion of the subject to measure a pulse wave with the at least one pulse wave sensor, the control unit configured to set a pressing force of the pressing member to a second pressing force lower than the first pressing force and higher than zero when there is body motion of the subject and interrupt measurement of a pulse wave.

In the present specification, “measurement site” refers to a site through which an artery passes. The measurement site may be, for example, an upper limb such as a wrist or an upper arm, or a lower limb such as an ankle or a thigh.

In addition, “belt” refers to a band-shaped member mounted around a measurement site regardless of the name. For example, instead of the belt, the name may be “band”, “cuff”, or the like.

In addition, the “width direction” of the belt corresponds to the longitudinal direction of the measurement site.

In addition, the “body motion” refers to the motion of the subject's body which brings significant variation in the pulse wave signal detected by at least one pulse wave sensor.

In addition, the “first pressing force” is the force of strength that can appropriately measure the pulse wave with at least one pulse wave sensor.

In addition, the “second pressing force” is the force of strength to the extent that an unnecessary physical load is not placed on the subject and to the extent that the position of at least one pulse wave sensor does not deviate from the measurement site as long as the body motion of the subject is not excessively violent.

In another aspect, a pulse wave measurement method of the present disclosure is a pulse wave measurement method includes:

using
a belt to be mounted around a measurement site of a subject,
at least one pulse wave sensor mounted on the belt, the at least one pulse wave sensor configured to detect a pulse wave of an artery passing through the measurement site,
a pressing member mounted on the belt, the pressing member configured to vary a pressing force to press the at least one pulse wave sensor against the measurement site, and
a body motion detection unit configured to detect presence or absence of body motion of the subject,

to measure a pulse wave of the measurement site, the pulse wave measurement method comprising:

setting a pressing force of the pressing member to a first pressing force when there is no body motion of the subject to measure a pulse wave with the at least one pulse wave sensor; and

setting a pressing force of the pressing member to a second pressing force lower than the first pressing force and higher than zero when there is body motion of the subject and interrupting measurement of a pulse wave.

### BRIEF DESCRIPTION OF DRAWINGS

The present invention will become more fully understood from the detailed description given hereinbelow and the accompanying drawings which are given by way of illustration only, and thus are not limitative of the present invention, and wherein:

FIG. 1 is a perspective view illustrating an appearance of a sphygmomanometer being a blood pressure measurement device including a pulse wave measurement device according to a first embodiment of the present invention.

FIG. 2 is a diagram schematically illustrating a cross section perpendicular to a longitudinal direction of a wrist in a state where the sphygmomanometer in FIG. 1 is mounted on the left wrist of the subject.

FIG. 3 is a planar layout diagram of impedance measurement electrodes constituting first and second pulse wave sensors in a state where the sphygmomanometer in FIG. 1 is mounted on the left wrist of the subject.

FIG. 4 is a diagram illustrating a block configuration of a control system of the sphygmomanometer in FIG. 1.

FIG. 5A is a diagram schematically illustrating a cross section along the longitudinal direction of the wrist in a state where the sphygmomanometer in FIG. 1 is mounted on the left wrist of the subject.

FIG. 5B is a diagram illustrating waveforms of first and second pulse wave signals respectively output from the first and second pulse wave sensors.

FIG. 6 is a diagram illustrating an operation flow when the sphygmomanometer in FIG. 1 performs blood pressure measurement by oscillometric method.

FIG. 7 is a diagram illustrating changes in a cuff pressure and a pulse wave signal according to the operation flow in FIG. 6.

FIG. 8 illustrates an operation flow when the sphygmomanometer executes a pulse wave measurement method of one embodiment to acquire pulse transit time (PTT) and to perform blood pressure measurement (estimation) based on the pulse transit time.

FIG. 9 is a graph illustrating a cuff pressure Pc set according to the presence or absence of body motion in the sphygmomanometer in FIG. 1.

FIG. 10 is a diagram illustrating a block configuration of the control system of a sphygmomanometer being a blood pressure measurement device including a pulse wave measurement device according to a second embodiment of the present invention.

FIG. 11 is a diagram schematically illustrating a cross section along the longitudinal direction of the wrist in a state where the sphygmomanometer in FIG. 10 is mounted on the left wrist of the subject.

FIG. 12 is a graph illustrating a cuff pressure Pc set according to the presence or absence of body motion in the sphygmomanometer in FIG. 10.

FIG. 13 is a diagram illustrating an example of a predetermined correspondence equation between pulse transit time and blood pressure.

FIG. 14 is a diagram illustrating another example of a predetermined correspondence equation between pulse transit time and blood pressure.

FIG. 15 is a diagram illustrating still another example of a predetermined correspondence equation between pulse transit time and blood pressure.

FIG. 16 is a diagram illustrating an equation representing a cross-correlation coefficient r between a data sequence {xᵢ} and a data sequence {yᵢ}.

### DESCRIPTION OF EMBODIMENTS

Hereinafter, embodiments of the present invention will be described in detail with reference to the drawings.

### First Embodiment

Hereinafter, a blood pressure measurement device including a pulse wave measurement device according to a first embodiment of the present invention will be described.

### (Configuration of Sphygmomanometer)

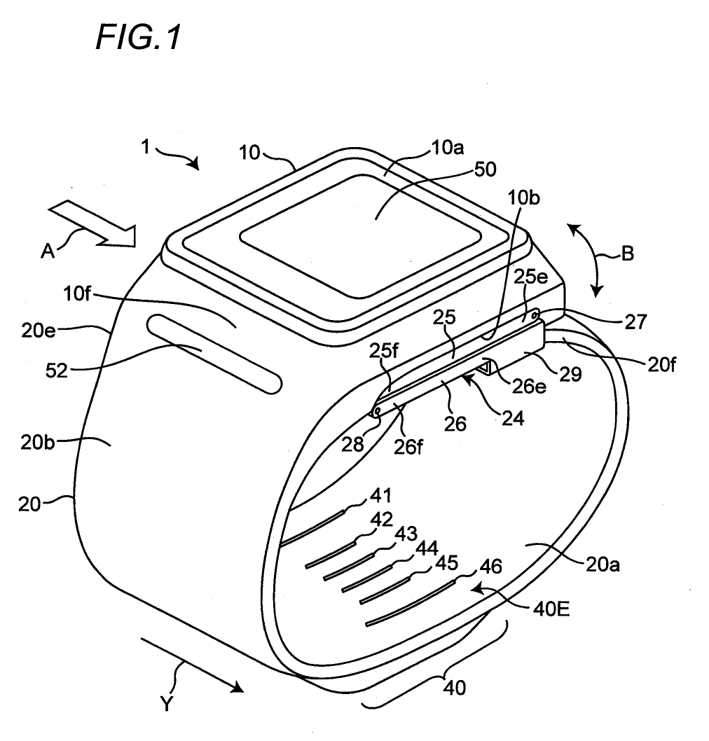

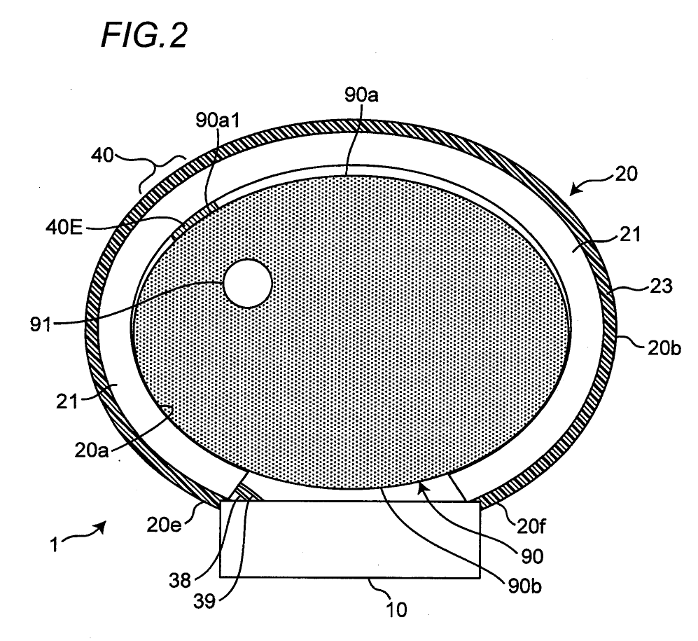

FIG. 1 illustrates an appearance of a wrist-type sphygmomanometer (the whole is denoted by reference numeral 1) being a blood pressure measurement device including a pulse wave measurement device according to the first embodiment of the present invention as viewed from an oblique direction. In addition, FIG. 2 schematically illustrates a cross section perpendicular to the longitudinal direction of the left wrist 90 in a state where the sphygmomanometer 1 is mounted on the left wrist 90 as a measurement site (hereinafter referred to as “mounted state”).

As illustrated in these drawings, the sphygmomanometer 1 roughly includes a belt 20 to be worn around a user's left wrist 90 and a main body 10 integrally attached to the belt 20.

As well understood from FIG. 1, the belt 20 has an elongated belt shape to surround the left wrist 90 along the circumferential direction, an inner peripheral surface 20a to be in contact with the left wrist 90, and an outer peripheral surface 20b on the opposite side of the inner peripheral surface 20a. The dimension (width dimension) in the width direction Y of the belt 20 is set to about 30 mm in this example.

The main body 10 is integrally provided at one end portion 20e of the belt 20 in the circumferential direction by integral molding in this example. It should be noted that the belt 20 and the main body 10 may be separately formed, and the main body 10 may be integrally attached to the belt 20 via an engaging member (for example, a hinge or the like). In this example, the site where the main body 10 is disposed is intended to correspond to the back side surface of the left wrist 90 (the surface on the back side of the hand) 90b in the mounted state (see FIG. 2). In FIG. 2, a radial artery 91 passing near the palmar surface (the surface on the palmar side) 90a in the left wrist 90 is illustrated.

As well understood from FIG. 1, the main body 10 has a three-dimensional shape having a thickness in a direction perpendicular to the outer peripheral surface 20b of the belt 20. The main body 10 is formed small and thin so as not to interfere with the daily activities of the user. In this example, the main body 10 has a truncated quadrangular pyramid-shaped contour projecting outward from the belt 20.

A display 50 serving as a display screen is provided on the top surface of the main body 10 (the surface on a side farthest from the measurement site) 10a. In addition, an operation unit 52 for inputting instructions from the user is provided along the side surface 10f of the main body 10 (side surface on the left front side in FIG. 1).

An impedance measurement unit 40 constituting at least one pulse wave sensor is provided in a site between one end 20e and the other end 20f in the circumferential direction of the belt 20. In the present embodiment, the case where the impedance measurement unit 40 constitutes first and second pulse wave sensors will be described. Of the belt 20, on the inner peripheral surface 20a of the site where the impedance measurement unit 40 is disposed, six plate-shaped (or sheet-shaped) electrodes 41 to 46 (all of which are referred to as “electrode group” and denoted by reference numeral 40E) are arranged in a state of being separated from each other in the width direction Y of the belt 20 (described in detail below). In this example, the site where the electrode group 40E is disposed is intended to correspond to the radial artery 91 of the left wrist 90 in the mounted state (see FIG. 2).

As illustrated in FIG. 1, the bottom surface of the main body 10 (the surface on the side closest to the measurement site) 10b and the end portion 20f of the belt 20 are connected by a threefold buckle 24. The buckle 24 includes a first plate-shaped member 25 disposed on the outer peripheral side and a second plate-shaped member 26 disposed on the inner peripheral side. One end portion 25e of the first plate-shaped member 25 is rotatably attached to the main body 10 via a coupling rod 27 extending along the width direction Y. The other end portion 25f of the first plate-shaped member 25 is rotatably attached to one end portion 26e of the second plate-shaped member 26 via a coupling rod 28 extending along the width direction Y. The other end portion 26f of the second plate-shaped member 26 is fixed near the end portion 20f of the belt 20 by the fixing portion 29. It should be noted that the attaching position of the fixing portion 29 in the circumferential direction of the belt 20 is variably set in advance in accordance with the circumferential length of the left wrist 90 of the user. Thus, the sphygmomanometer 1 (belt 20) is formed in a substantially annular shape as a whole, and the bottom surface 10b of the main body 10 and the end portion 20f of the belt 20 can be opened and closed in the arrow B direction by the buckle 24.

When mounting the sphygmomanometer 1 on the left wrist 90, the user inserts the left hand into the belt 20 in the direction indicated by the arrow A in FIG. 1 with the buckle 24 open and the diameter of the ring of the belt 20 increased. Then, as illustrated in FIG. 2, the user adjusts the angular position of the belt 20 around the left wrist 90 to position the impedance measurement unit 40 of the belt 20 on the radial artery 91 passing through the left wrist 90. Thus, the electrode group 40E of the impedance measurement unit 40 abuts on a portion 90a1 corresponding to the radial artery 91 on the palmar surface 90a of the left wrist 90. In this state, the user closes and fixes the buckle 24. Thus, the user wears the sphygmomanometer 1 (belt 20) on the left wrist 90.

As illustrated in FIG. 2, in this example, the belt 20 includes a strip 23 forming the outer peripheral surface 20b and a pressing cuff 21 as a pressing member attached along the inner peripheral surface of the strip 23. The strip 23 is, in this example, made of a plastic material that is flexible in the thickness direction and substantially non-stretchable in the circumferential direction (longitudinal direction). In this example, the pressing cuff 21 is configured as a fluid bag by facing two stretchable polyurethane sheets in the thickness direction and welding their peripheral portions. The electrode group 40E of the impedance measurement unit 40 is disposed at a site corresponding to the radial artery 91 of the left wrist 90 on the inner peripheral surface 20a of the pressing cuff 21 (belt 20) as described above.

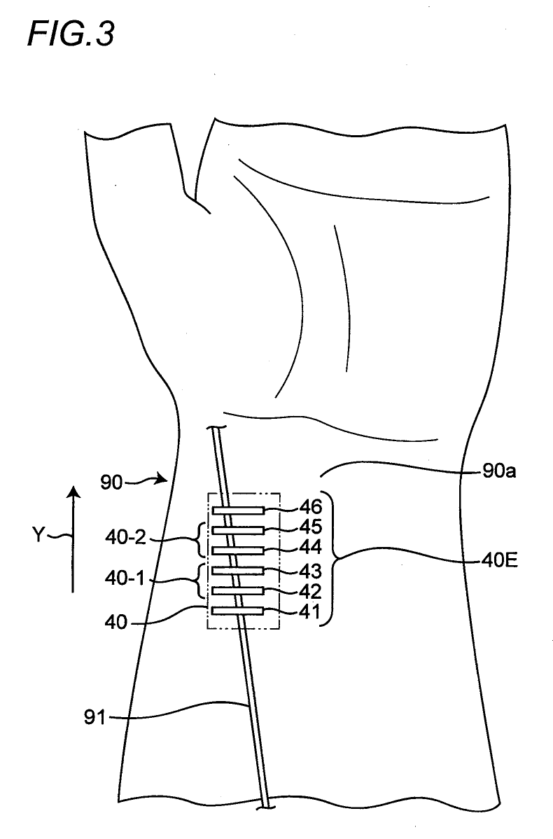

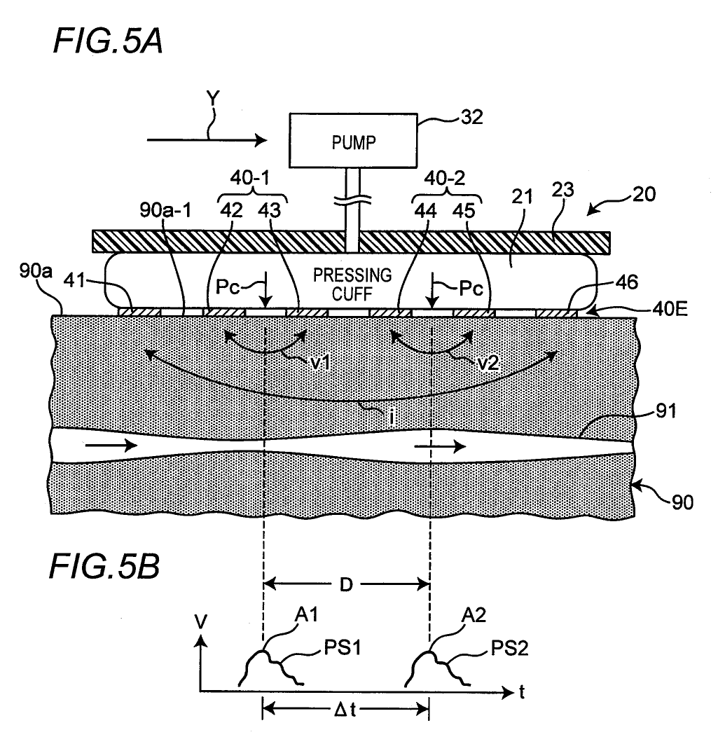

As illustrated in FIG. 3, in the mounted state, the electrode group 40E of the impedance measurement unit 40 is aligned along the longitudinal direction of the wrist (corresponding to the width direction Y of the belt 20) according to the radial artery 91 of the left wrist 90. The electrode group 40E includes a current electrode pair 41 and 46 for energization disposed on both sides in the width direction Y, a first detection electrode pair 42 and 43 forming a first pulse wave sensor 40-1 for voltage detection disposed between the current electrode pair 41 and 46, and a second detection electrode pair 44 and 45 forming a second pulse wave sensor 40-2. With respect to the first detection electrode pair 42 and 43, the second detection electrode pair 44 and 45 is disposed according to the portion on the more downstream side of the blood flow of the radial artery 91. In the width direction Y, the distance D between the center of the first detection electrode pair 42 and 43 and the center of the second detection electrode pair 44 and 45 (see FIG. 5A) is set to 20 mm in this example. This distance D corresponds to a substantial space between the first pulse wave sensor 40-1 and the second pulse wave sensor 40-2. In addition, in the width direction Y, the space between the first detection electrode pair 42 and 43 and the space between the second detection electrode pair 44 and 45 are both set to 2 mm in this example.

This electrode group 40E can be configured to be flat. Therefore, in the sphygmomanometer 1, the belt 20 can be configured to be thin as a whole.

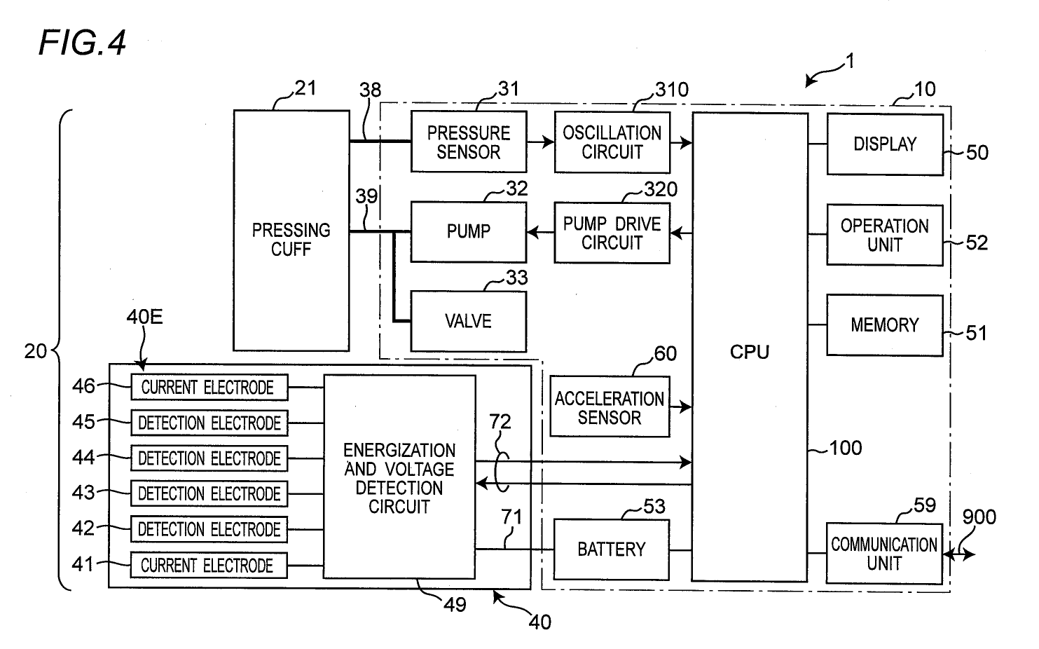

FIG. 4 illustrates a block configuration of a control system of the sphygmomanometer 1. In addition to the display 50 and the operation unit 52 described above, the main body 10 of the sphygmomanometer 1 mounts a central processing unit (CPU) 100 as a control unit, a memory 51 as a storage unit, a communication unit 59, a pressure sensor 31, a pump 32, a valve 33, an oscillation circuit 310 for converting the output from the pressure sensor 31 into a frequency, a pump drive circuit 320 for driving the pump 32, and an acceleration sensor 60 for measuring acceleration applied to the sphygmomanometer 1. Furthermore, in addition to the electrode group 40E described above, the impedance measurement unit 40 mounts an energization and voltage detection circuit 49.

The display 50 includes an organic electro luminescence (EL) display in this example, and displays information related to blood pressure measurement such as blood pressure measurement results and other information in accordance with a control signal from the CPU 100. It should be noted that the display 50 is not limited to the organic EL display, and may include another type of display such as a liquid crystal display (LCD).

The operation unit 52 includes a push switch in this example, and inputs an operation signal corresponding to the user's instructions to start or stop blood pressure measurement into the CPU 100. It should be noted that the operation unit 52 is not limited to the push switch, and may be, for example, a pressure-sensitive (resistive) or proximity (capacitive) touch panel switch. In addition, the operation unit 52 may include a microphone (not shown) to input a blood pressure measurement start instructions in response to the user's voice.

The memory 51 non-transitorily stores data of a program for controlling the sphygmomanometer 1, data used for controlling the sphygmomanometer 1, setting data for setting various functions of the sphygmomanometer 1, data of measurement results of blood pressure values, and the like. In addition, the memory 51 is used as a work memory or the like when a program is executed.

The CPU 100 executes various functions as a control unit in accordance with a program for controlling the sphygmomanometer 1 stored in the memory 51. For example, when blood pressure measurement is performed by oscillometric method, the CPU 100 drives the pump 32 (and the valve 33) based on a signal from the pressure sensor 31 in response to instructions to start blood pressure measurement from the operation unit 52. In addition, the CPU 100 calculates the blood pressure value based on the signal from the pressure sensor 31 in this example.

The communication unit 59 is controlled by the CPU 100 to transmit predetermined information to an external device via the network 900, receives information from an external device via the network 900, and delivers the information to the CPU 100. The communication via the network 900 may be wireless or wired. In this embodiment, the network 900 is the Internet, but is not limited thereto, and may be another type of network such as a hospital local area network (LAN), or may be one-to-one communication using a USB cable or the like. The communication unit 59 may include a micro USB connector.

The pump 32 and the valve 33 are connected to the pressing cuff 21 via the air pipe 39, and the pressure sensor 31 is connected to the pressing cuff 21 via the air pipe 38. It should be noted that the air pipes 39 and 38 may be one common pipe. The pressure sensor 31 detects the pressure in the pressing cuff 21 via the air pipe 38. The pump 32 includes a piezoelectric pump in this example and supplies air as a fluid for pressurization to the pressing cuff 21 through the air pipe 39 in order to raise the pressure in the pressing cuff 21 (cuff pressure). The valve 33 is mounted on the pump 32, and is configured to be controlled in opening/closing as the pump 32 is turned on/off. That is, when the pump 32 is turned on, the valve 33 closes and air is filled into the pressing cuff 21, while when the pump 32 is turned off, the valve 33 opens and the air in the pressing cuff 21 is discharged into the atmosphere through the air pipe 39. It should be noted that the valve 33 has a function of a check valve so that the discharged air does not flow back. The pump drive circuit 320 drives the pump 32 based on a control signal supplied from the CPU 100.

The pressure sensor 31 is a piezoresistive pressure sensor in this example, and detects the pressure of the belt 20 (pressing cuff 21), a pressure with the atmospheric pressure as a reference (zero) in this example, through the air pipe 38 to output the detected result as a time-series signal. The oscillation circuit 310 oscillates based on an electrical signal value based on a change in electrical resistance due to the piezoresistive effect from the pressure sensor 31, and outputs a frequency signal having a frequency corresponding to the electrical signal value of the pressure sensor 31 to the CPU 100. In this example, the output of pressure sensor 31 is used for controlling the pressure of the pressing cuff 21, and for calculating the blood pressure value (including systolic blood pressure (SBP) and diastolic blood pressure (DBP)) by oscillometric method.

The acceleration sensor 60 measures the acceleration applied to the sphygmomanometer 1 to work as a body motion detection unit for detecting the presence or absence of body motion of the subject.

The battery 53 supplies power to elements mounted on the main body 10, in this example, to each element of the CPU 100, the pressure sensor 31, the pump 32, the valve 33, the display 50, the memory 51, the communication unit 59, the oscillation circuit 310, the pump drive circuit 320, and the acceleration sensor 60. In addition, the battery 53 also supplies power to the energization and voltage detection circuit 49 of the impedance measurement unit 40 through the wiring line 71. This wiring line 71 is provided to extend between the main body 10 and the impedance measurement unit 40 along the circumferential direction of the belt 20 in a state of being sandwiched between the strip 23 of the belt 20 and the pressing cuff 21 together with the signal wiring line 72.

The energization and voltage detection circuit 49 of the impedance measurement unit 40 is controlled by the CPU 100, and supplies a high frequency constant current i having a frequency of 50 kHz and a current value of 1 mA, in this example, between the current electrode pair 41 and 46 disposed on both sides in the longitudinal direction of the wrist (corresponding to the width direction Y of the belt 20) during the operation, as illustrated in FIG. 5A. In this state, the energization and voltage detection circuit 49 detects a voltage signal v1 between the first detection electrode pair 42 and 43 forming the first pulse wave sensor 40-1 and a voltage signal v2 between the second detection electrode pair 44 and 45 forming the second pulse wave sensor 40-2. These voltage signals v1 and v2 respectively represent the change in the electrical impedance due to the pulse wave of the blood flow of the radial artery 91 in the portions where the first pulse wave sensor 40-1 and the second pulse wave sensor 40-2 face on the palmar surface 90a of the left wrist 90 (impedance system). The energization and voltage detection circuit 49 rectifies, amplifies, and filters these voltage signals v1 and v2 to output a first pulse wave signal PS1 and a second pulse wave signal PS2 having mountain-shaped waveforms in time series as illustrated in FIG. 5B. In this example, the voltage signals v1 and v2 are approximately 1 mV. In addition, the respective peaks A1 and A2 of the first pulse wave signal PS1 and the second pulse wave signal PS2 are approximately 1 volt in this example.

It should be noted that assuming that the pulse wave velocity (PWV) of the blood flow of the radial artery 91 is in the range of 1000 cm/s to 2000 cm/s, since the substantial space D between the first pulse wave sensor 40-1 and the second pulse wave sensor 40-2 is 20 mm, the time difference Δt between the first pulse wave signal PS1 and the second pulse wave signal PS2 is in the range of 1.0 ms to 2.0 ms.

### (Operation of Blood Pressure Measurement by Oscillometric Method)

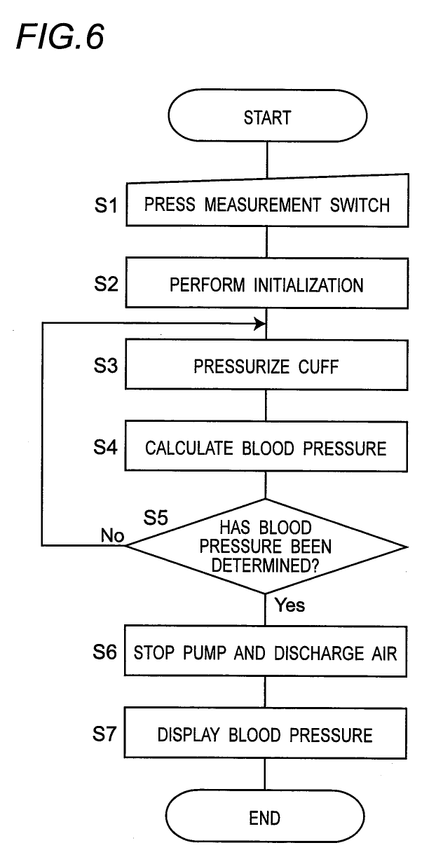

FIG. 6 illustrates an operation flow when the sphygmomanometer 1 performs blood pressure measurement by oscillometric method.

When the user gives an instruction to measure blood pressure by oscillometric method with the push switch of the operation unit 52 provided in the main body 10 (step S1), the CPU 100 starts operation to initialize the processing memory area (step S2). In addition, the CPU 100 turns off the pump 32 via the pump drive circuit 320, opens the valve 33, and discharges the air in the pressing cuff 21. Subsequently, the current output value of the pressure sensor 31 is set as a value corresponding to the atmospheric pressure (0 mmHg adjustment).

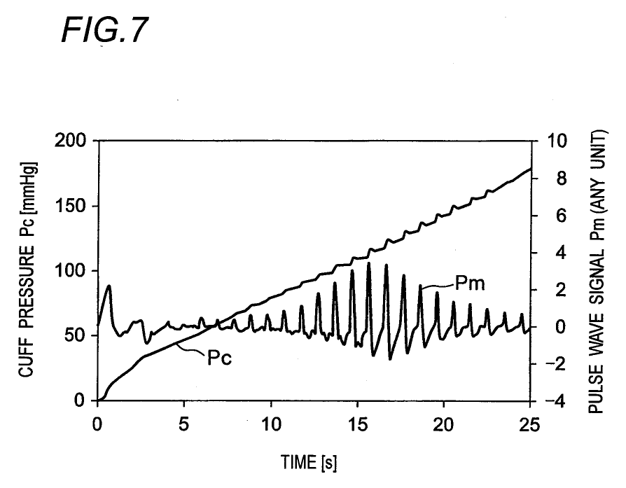

Subsequently, the CPU 100 works as a pressure control unit and drives the pump 32 via the pump drive circuit 320 to send air to the pressing cuff 21, which closes the valve 33 to inflate the pressing cuff 21, gradually pressurizing the cuff pressure Pc (see FIG. 7) (step S3 in FIG. 6).

In this pressurization process, the CPU 100 monitors the cuff pressure Pc with the pressure sensor 31 in order to calculate the blood pressure value, and acquires, as a pulse wave signal Pm as illustrated in FIG. 7, the fluctuation component of the arterial volume generated in the radial artery 91 of the left wrist 90 as the measurement site.

Next, in step S4 in FIG. 6, the CPU 100 acts as a second blood pressure calculation unit, and applies a known algorithm by oscillometric method based on the pulse wave signal Pm acquired at this time to attempt the calculation of blood pressure values (systolic blood pressure SBP and diastolic blood pressure DBP).

At this time, if the blood pressure value cannot be calculated yet because of insufficient data (NO in step S5), unless the cuff pressure Pc reaches the upper limit pressure (for safety, for example, 300 mmHg is predetermined), the processing of steps S3 to S5 is repeated.

If the blood pressure value can be calculated in this manner (YES in step S5), the CPU 100 stops the pump 32, opens the valve 33, and discharges the air in the pressing cuff 21 (step S6). Then, lastly, the measurement result of the blood pressure value is displayed on the display 50 and recorded in the memory 51 (step S7).

It should be noted that the calculation of the blood pressure value may be performed not only in the pressurization process, but also in the depressurization process.

### (Operation of Blood Pressure Measurement Based on Pulse Transit Time)

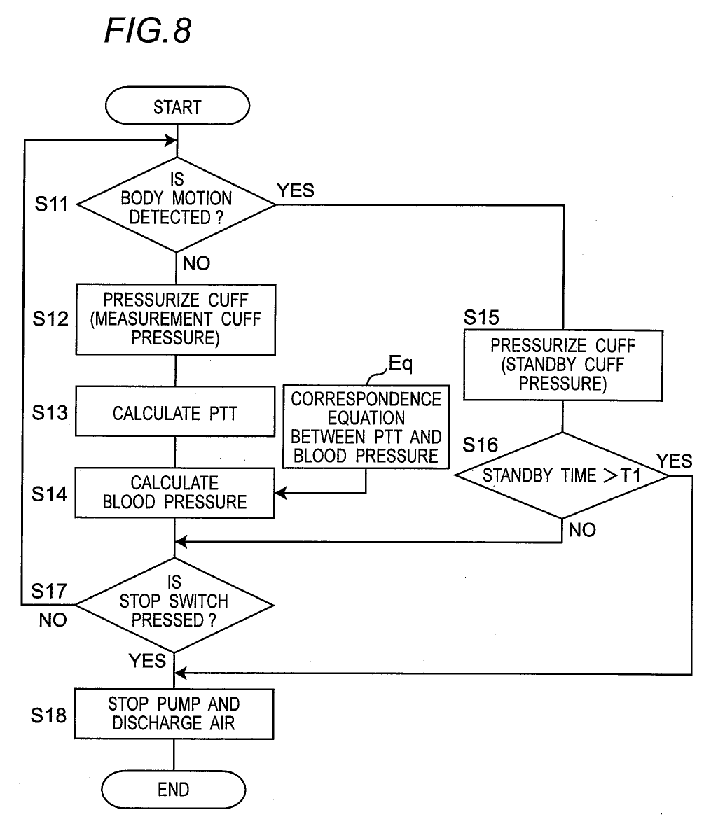

FIG. 8 illustrates an operation flow when the sphygmomanometer 1 executes a pulse wave measurement method of one embodiment to acquire pulse transit time (PTT) and to perform blood pressure measurement (estimation) based on the pulse transit time.

When the user gives an instruction to perform the PTT-based blood pressure measurement with a push switch of the operation unit 52 provided on the main body 10, the CPU 100 starts operation. First, the CPU 100 detects the presence or absence of body motion of the subject by using the acceleration sensor 60 (step S11 in FIG. 8).

When there is no body motion of the subject (NO in step S11 in FIG. 8), the CPU 100 sets the pressing force of the pressing cuff 21 to a predetermined measurement cuff pressure (first pressing force) (step S12 in FIG. 8). The method for determining the measurement cuff pressure (first pressing force) will be described below. The CPU 100 drives the pump 32 via the pump drive circuit 320 to send air to the pressing cuff 21, which closes the valve 33 to inflate the pressing cuff 21, pressurizing the cuff pressure Pc (see FIG. 5A) to the measurement cuff pressure.

Next, the CPU 100 measures the first and second pulse wave signals PS1 and PS2 with the first pulse wave sensor 40-1 and the second pulse wave sensor 40-2, and acquires a time difference Δt between the first and second pulse wave signals PS1 and PS2 (see FIG. 5B) as pulse transit time (PTT) (step S13 in FIG. 8). More specifically, in this example, a time difference Δt between the peak A1 of the first pulse wave signal PS1 and the peak A2 of the second pulse wave signal PS2 is acquired as pulse transit time (PTT).

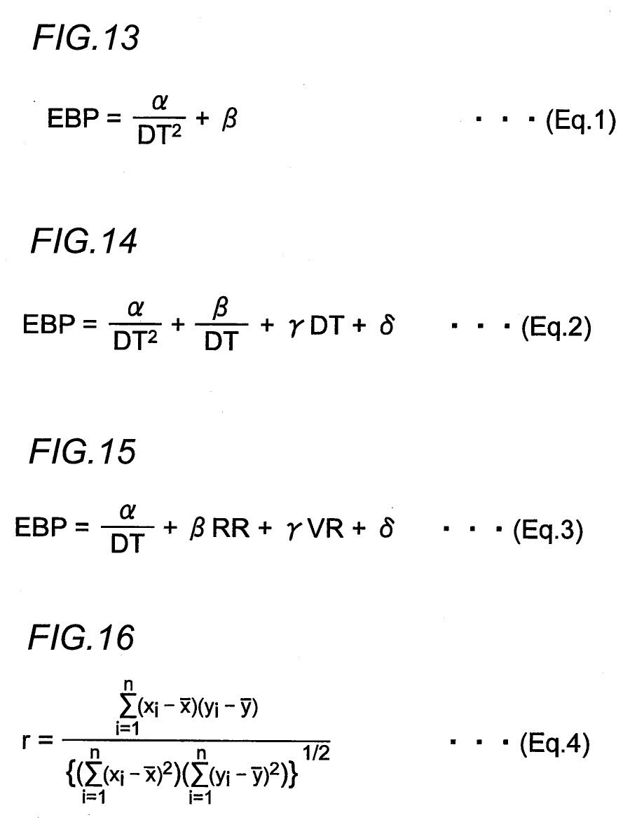

Next, the CPU 100 works as a first blood pressure calculation unit, and calculates (estimates) the blood pressure based on the pulse transit time (PTT) acquired in step S13 by using the predetermined correspondence equation Eq between pulse transit time and blood pressure (step S14 in FIG. 8). Here, when pulse transit time is represented as DT and blood pressure is represented as EBP, the predetermined correspondence equation Eq between pulse transit time and blood pressure is provided as a known fractional function including the term of 1/DT², for example, as shown in the equation (Eq. 1) in FIG. 13 (see, for example, JP H10-201724 A). In the equation (Eq. 1), each of α and β represents a known coefficient or constant.

The measurement result of the blood pressure value is displayed on the display 50 and recorded in the memory 51.

On the other hand, when there is body motion of the subject (YES in step S11 in FIG. 8), the CPU 100 sets the pressing force of the pressing cuff 21 to a standby cuff pressure (a second pressing force lower than the first pressing force and higher than zero), and interrupts the measurement of the pulse wave (step S15 in FIG. 8). When interrupting the measurement of the pulse wave immediately after the start of the operation flow in FIG. 1, the CPU 100 drives the pump 32 via the pump drive circuit 320 to send air to the pressing cuff 21, which closes the valve 33 to inflate the pressing cuff 21, pressurizing the cuff pressure to the standby cuff pressure. On the other hand, if the pressing force of the pressing cuff 21 is once set to the measurement cuff pressure, and then the pulse wave measurement is interrupted, the CPU 100 stops the pump 32 via the pump drive circuit 320, thereby opening the valve 33 to reduce the cuff pressure Pc to the standby cuff pressure. Then, when the cuff pressure Pc reaches the standby cuff pressure, the CPU 100 temporarily operates the pump 32 again via the pump drive circuit 320, thereby closing the valve 33. The CPU 100 may display on the display 50 that the measurement of the pulse wave is interrupted due to detecting the body motion of the subject.

Next, the CPU 100 determines whether the standby time after interrupting the measurement of the pulse wave (that is, the standby time after setting the standby cuff pressure (second pressing force)) exceeds a threshold value T1 having a predetermined length (step S16 in FIG. 8). If NO in step S16 in FIG. 8, the CPU 100 proceeds to step S17, and as long as body motion of the subject is detected in step S11 (YES in step S11 in FIG. 8), the CPU 100 repeats the loop of steps S11, 15, S16, and S17. If the state of YES is continued in step S11 in FIG. 8 (that is, if the loop of steps S11, 15, S16, and S17 is repeated), the standby time after interrupting pulse wave measurement is the total value thereof. When the standby time after interrupting the measurement of the pulse wave exceeds the threshold value T1 (YES in step S16 in FIG. 8), the CPU 100 stops the pump 32, opens the valve 33 to discharge the air in the pressing cuff 21, and sets the pressing force of the pressing cuff 21 to zero (step S18 in FIG. 8).

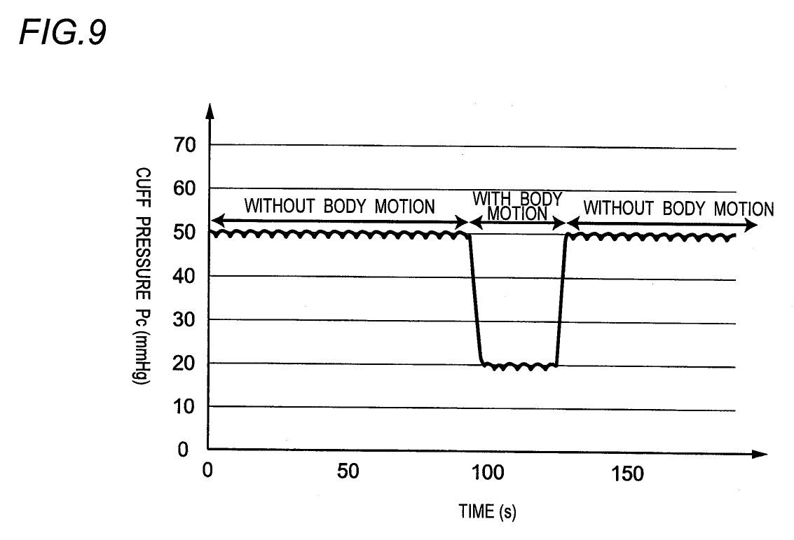

In this example, if measurement stop is not instructed by the push switch of the operation unit 52 in step S17 in FIG. 8 (NO in step S17 in FIG. 8), the CPU 100 returns to step S11 to repeat any one of steps S12 to S14 for setting the measurement cuff pressure and steps S15 to S16 for setting the standby cuff pressure according to the presence or absence of body motion of the subject. FIG. 9 is a graph illustrating a cuff pressure Pc set according to the presence or absence of body motion in the sphygmomanometer in FIG. 1. When there is no body motion of the subject, the cuff pressure Pc is set to, for example, 50 mmHg, and when there is body motion of the subject, the cuff pressure Pc is set to, for example, 20 mmHg. After interrupting the measurement of the pulse wave (YES in step S11 in FIG. 8), and when a state where there is body motion of the subject is transitioned to a state where there is no body motion of the subject (NO in step S11 in FIG. 8), the CPU 100 returns the pressing force of the pressing cuff 21 from the second pressing force to the first pressing force to resume measurement of the pulse wave. Every time executing steps S12 to S14, the CPU 100 updates and displays the measurement result of the blood pressure value on the display 50 and accumulates and records the measurement result of the blood pressure value in the memory 51.

If the user gives an instruction to stop measurement with the push switch of the operation unit 52 provided on the main body 10 (YES in step S17 in FIG. 8), the CPU 100 stops the pump 32, opens the valve 33, discharges the air in the pressing cuff 21, and ends the measurement operation (step S18).

According to the sphygmomanometer 1, it is possible to control the pressing force on the measurement site by a novel control method in consideration of the body motion of the subject, and to improve the convenience of the subject. According to the sphygmomanometer 1, when there is body motion of the subject, reducing the pressing force of the pressing cuff allows the physical burden on the subject to be relieved. In addition, according to the sphygmomanometer 1, when the pressing force of the pressing cuff is reduced, setting the pressing force higher than zero allows the positional deviation of the pulse wave sensors 40-1 and 40-2 to be reduced and the pressurization time when measurement is resumed to be shortened.

According to the sphygmomanometer 1, the blood pressure measurement based on the pulse transit time (PTT) allows blood pressure to be measured continuously over a long period of time with a reduced physical burden on the user.

In addition, according to the sphygmomanometer 1, the blood pressure measurement (estimation) based on pulse transit time and the blood pressure measurement by oscillometric method can be performed by an integrated device. Therefore, the convenience of the user can be enhanced.

### (Determination of First Pressing Force)

The measurement cuff pressure (first pressing force) set in step S12 in FIG. 8 is determined, for example, as follows.

According to experiments by the inventor, it has been found that when the pressing force of the first pulse wave sensor 40-1 (including the first detection electrode pair 42 and 43) and the second pulse wave sensor 40-2 (including the second detection electrode pair 44 and 45) on the left wrist 90 as the measurement site (equal to the cuff pressure Pc by the pressing cuff 21) gradually increases from zero, the cross-correlation coefficient r between the waveforms of the first and second pulse wave signals PS1 and PS2 gradually increases along with that, indicates the maximum value rmax, and then gradually decreases. This operation flow is based on the idea that a range in which the cross-correlation coefficient r exceeds a predetermined threshold value Th (in this example, Th=0.99) is an appropriate range of the pressing force (this is referred to as an “appropriate pressing range”).

In order to determine the first pressing force, first, the CPU 100 drives the pump 32 via the pump drive circuit 320 to send air to the pressing cuff 21, which closes the valve 33 to inflate the pressing cuff 21, gradually pressurizing the cuff pressure Pc (see FIG. 5A). In this example, the cuff pressure Pc is continuously increased at a constant rate (=5 mmHg/s). It should be noted that the cuff pressure Pc may be increased stepwise so that the time for calculating the cross-correlation coefficient r described next is easily secured.

In this pressurization process, the CPU 100 acquires first and second pulse wave signals PS1 and PS2 respectively output in time series by the first pulse wave sensor 40-1 and the second pulse wave sensor 40-2, and calculates the cross-correlation coefficient r between the waveforms of the first and second pulse wave signals PS1 and PS2 in real time.

Along with that, the CPU 100 determines whether the calculated cross-correlation coefficient r exceeds a predetermined threshold value Th (=0.99). Here, if the cross-correlation coefficient r is not more than the threshold value Th, the CPU 100 repeats the pressurization of the cuff pressure Pc and the calculation of the cross-correlation coefficient r until the cross-correlation coefficient r exceeds the threshold value Th. Then, if the cross-correlation coefficient r exceeds the threshold value Th, the CPU 100 stops the pump 32 and sets the cuff pressure Pc to a value at that time, that is, a value when the cross-correlation coefficient r exceeds the threshold value Th.

Using the measurement cuff pressure (first pressing force) determined in this manner allows the measurement accuracy of the pulse transit time to be enhanced. In addition, since the cuff pressure Pc is set to a value when the cross-correlation coefficient r exceeds the threshold value Th, the pulse transit time can be acquired without unnecessarily increasing the cuff pressure Pc. Thus, the physical burden on the user can be reduced.

### Second Embodiment

Hereinafter, a blood pressure measurement device including a pulse wave measurement device according to a second embodiment of the present invention will be described.

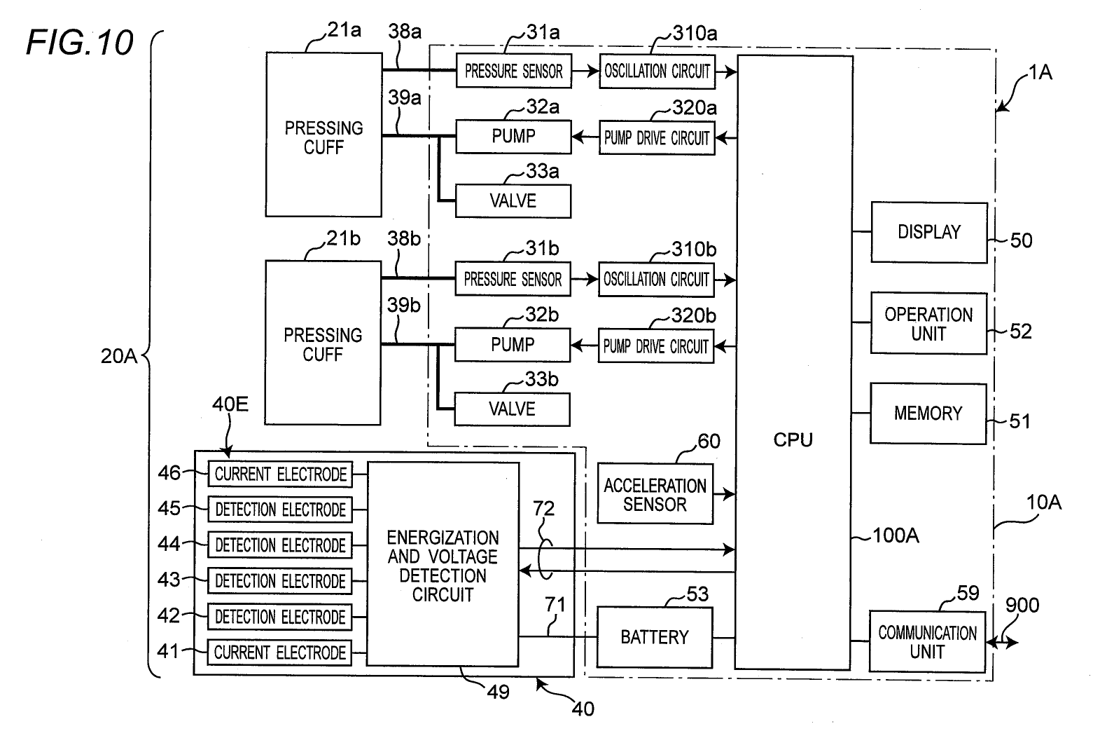

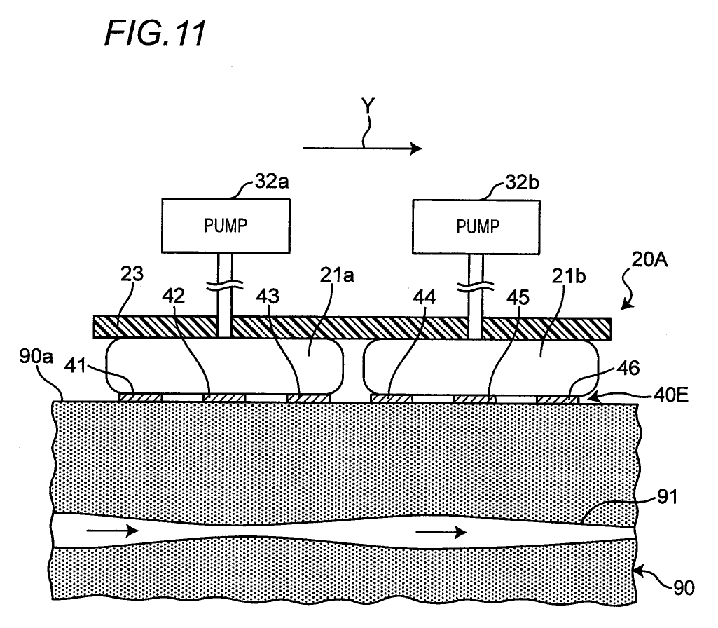

FIG. 10 is a diagram illustrating a block configuration of the control system of a sphygmomanometer 1A being a blood pressure measurement device including a pulse wave measurement device according to a second embodiment of the present invention. FIG. 11 is a diagram schematically illustrating a cross section along the longitudinal direction of the wrist in a state where the sphygmomanometer in FIG. 10 is mounted on the left wrist of the subject.

The sphygmomanometer 1A includes a main body 10A and a belt 20A.

Instead of one system of the pressure sensor 31, the pump 32, the valve 33, the oscillation circuit 310, the pump drive circuit 320, and the CPU 100 for controlling them included in the main body 10 in FIG. 4, the main body 10A in FIG. 10 includes two systems of pressure sensors 31a and 31b, pumps 32a and 32b, valves 33a and 33b, oscillation circuits 310a and 310b, pump drive circuits 320a and 320b, and a CPU 100A for controlling them. The pressure sensors 31a and 31b, the pumps 32a and 32b, the valves 33a and 33b, the oscillation circuits 310a and 310b, and the pump drive circuits 320a and 320b in FIG. 10 are respectively configured in the same manner as the pressure sensor 31, the pump 32, the valve 33, the oscillation circuit 310, and the pump drive circuit 320 in FIG. 4.

The belt 20A in FIG. 10 includes two pressing cuffs 21a and 21b instead of one pressing cuff 21 of the belt 20 in FIG. 4. Each of the pressing cuffs 21a and 21b in FIG. 10 is configured in the same manner as the pressing cuff 21 in FIG. 4. The pressing cuff 21a is connected to the pressure sensor 31a and the pump 32a via the air pipes 38a and 39a. The pressing cuff 21b is connected to the pressure sensor 31b and the pump 32b via the air pipes 38b and 39b.

The other components of the sphygmomanometer 1A in FIG. 10 are configured in the same manner as the corresponding components of the sphygmomanometer 1 in FIG. 4.

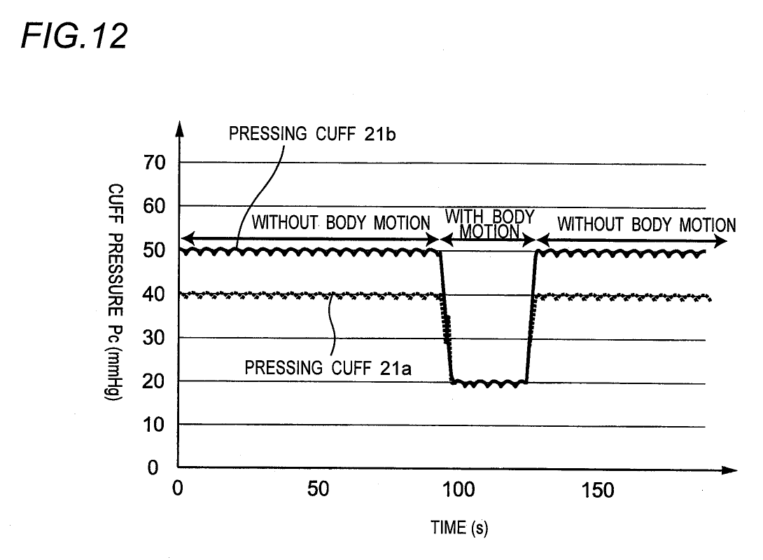

The sphygmomanometer 1A in FIG. 10 includes two systems of pumps 32a and 32b, thereby allowing the first pulse wave sensor (detection electrodes 42 and 43) and the second pulse wave sensor (detection electrodes 44 and 45) to be pressed with individual pressing forces (cuff pressure). The CPU 100 sets the first pressing force (cuff pressure) of the pressing cuffs 21a and 21b to individual values with respect to the first pulse wave sensor and the second pulse wave sensor. FIG. 12 is a graph illustrating a cuff pressure Pc set according to the presence or absence of body motion in the sphygmomanometer in FIG. 10. When there is no body motion of the subject, the cuff pressure Pc of the pressing cuff 21a is set to, for example, 40 mmHg, and when there is body motion of the subject, the cuff pressure Pc of the pressing cuff 21a is set to, for example, 20 mmHg. When there is no body motion of the subject, the cuff pressure Pc of the pressing cuff 21b is set to, for example, 50 mmHg, and when there is body motion of the subject, the cuff pressure Pc of the pressing cuff 21b is set to, for example, 20 mmHg.

The cuff pressure Pc of the pressing cuffs 21a and 21b as the first pressing force is, for example, set to a value at which the cross-correlation coefficient of the first and second pulse wave signals respectively output in time series by the first and second pulse wave sensors exceeds a predetermined threshold value. Setting the first pressing force (cuff pressure) of the pressing cuffs 21a and 21b to individual values easily brings the cross-correlation coefficient close to 1, and therefore, easily improves the measurement accuracy of the pulse wave and the blood pressure.

The cuff pressures Pc of the pressing cuffs 21a and 21b when there is body motion of the subject may be the same value or different values.

### (Modification)

In the above example, the acceleration sensor 60 is used to detect the presence or absence of body motion of the subject, but instead, for example, the pressure sensor 31 may be used to detect a change in cuff pressure caused by body motion of the subject. Both acceleration and a change in cuff pressure may be used to detect the presence or absence of body motion of the subject.

In the above example, the presence or absence of body motion of the subject is determined only in step S11 in FIG. 8, but instead, during the execution of steps S12 to S14, the presence or absence of body motion of the subject may always be determined, and when there is body motion of the subject, steps S12 to S14 may be interrupted and the process may proceed to step S15.

In addition, in the above example, in step S14 in FIG. 8, the equation (Eq. 1) in FIG. 13 is used as the correspondence equation Eq between pulse transit time and blood pressure so that the blood pressure is calculated (estimated) based on the pulse transit time (PTT). However, the present invention is not limited thereto. As a correspondence equation Eq between pulse transit time and blood pressure, where the pulse transit time is denoted by DT and the blood pressure is denoted by EBP, for example, as illustrated in the equation (Eq. 2) in FIG. 14, in addition to the term of 1/DT², an equation including the term of 1/DT and the term of DT may be used. In the equation (Eq. 2), each of α, β, γ, and δ represents a known coefficient or a constant.

Furthermore, for example, as illustrated in the equation (Eq. 3) in FIG. 15, an equation including the term of 1/DT, the term of the cardiac cycle RR, and the term of the plethysmogram area ratio VR may be used (see, for example, JP 2000-33078 A). In the equation (Eq. 3), each of α, γ, and δ represents a known coefficient or a constant. It should be noted that, in this case, the CPU 100 calculates the cardiac cycle RR and the plethysmogram area ratio VR based on the pulse wave signals PS1 and PS2.

The blood pressure can be measured in the same manner as in the case of using equation (Eq. 1) also in the case of using these equations (Eq. 2) and (Eq. 3) as the correspondence equation Eq between pulse transit time and blood pressure. Naturally, correspondence equations other than these equations (Eq. 1), (Eq. 2), and (Eq. 3) may be used.

In the above embodiment, the first pulse wave sensor 40-1 and the second pulse wave sensor 40-2 detect the pulse wave of the artery (radial artery 91) passing through the measurement site (left wrist 90) as a change in impedance (impedance system). However, the present invention is not limited thereto. Each of the first and second pulse wave sensors may include a light emitting element for applying light toward an artery passing through a corresponding portion of the measurement site and a light receiving element for receiving the reflected light (or transmitted light) of the light, and may detect a pulse wave of the artery as a change in volume (photoelectric system). Alternatively, each of the first and second pulse wave sensors may include a piezoelectric sensor abutted on the measurement site, and may detect the strain due to the pressure of the artery passing through the corresponding portion of the measurement site as a change in electrical resistance (piezoelectric system). Furthermore, each of the first and second pulse wave sensors may include a transmission element for transmitting a radio wave (transmission wave) toward an artery passing through a corresponding portion of the measurement site and a reception element for receiving the reflected wave of the radio wave, and may detect a change in the distance between the artery and the sensor due to the pulse wave of the artery as a phase shift between the transmission wave and the reflected wave (radio wave irradiation system).

Although the above embodiments describe the case where the sphygmomanometer in FIG. 1 performs blood pressure measurement (estimation) based on pulse transit time, the processing of controlling the pressing force on the measurement site in consideration of the body motion of the subject is applicable to any case of detecting a pulse wave by using at least one pulse wave sensor.

In addition, in the above embodiment, the sphygmomanometer 1 is intended to be mounted on the left wrist 90 as a measurement site. However, the present invention is not limited thereto. The measurement site has only to be a site where an artery passes through, may be an upper limb such as an upper arm other than the wrist, and may be a lower limb such as an ankle or thigh.

In addition, in the above embodiments, the CPU 100 mounted on the sphygmomanometer 1 is assumed to work as a body motion detection unit, a control unit, and first and second blood pressure calculation units to perform blood pressure measurement by oscillometric method (operation flow in FIG. 6) and blood pressure measurement (estimation) based on pulse wave measurement and PTT (operation flow in FIG. 8). However, the present invention is not limited thereto. For example, a substantial computer device such as a smartphone provided outside the sphygmomanometer 1 may work as a body motion detection unit, a control unit, and first and second blood pressure calculation units to cause, via the network 900, the sphygmomanometer 1 to perform blood pressure measurement by oscillometric method (operation flow in FIG. 6) and blood pressure measurement (estimation) based on pulse wave measurement and PTT (operation flow in FIG. 8).

As described above, a pulse wave measurement device of the present disclosure comprises:

a belt to be mounted around a measurement site of a subject;

at least one pulse wave sensor mounted on the belt, the at least one pulse wave sensor configured to detect a pulse wave of an artery passing through the measurement site;

a pressing member mounted on the belt, the pressing member configured to vary a pressing force to press the at least one pulse wave sensor against the measurement site;

a body motion detection unit configured to detect presence or absence of body motion of the subject; and

a control unit configured to set a pressing force of the pressing member to a first pressing force when there is no body motion of the subject to measure a pulse wave with the at least one pulse wave sensor, the control unit configured to set a pressing force of the pressing member to a second pressing force lower than the first pressing force and higher than zero when there is body motion of the subject and interrupt measurement of a pulse wave.

In the present specification, “measurement site” refers to a site through which an artery passes. The measurement site may be, for example, an upper limb such as a wrist or an upper arm, or a lower limb such as an ankle or a thigh.

In addition, “belt” refers to a band-shaped member mounted around a measurement site regardless of the name. For example, instead of the belt, the name may be “band”, “cuff”, or the like.

In addition, the “width direction” of the belt corresponds to the longitudinal direction of the measurement site.

In addition, the “body motion” refers to the motion of the subject's body which brings significant variation in the pulse wave signal detected by at least one pulse wave sensor.

In addition, the “first pressing force” is the force of strength that can appropriately measure the pulse wave with at least one pulse wave sensor.

In addition, the “second pressing force” is the force of strength to the extent that an unnecessary physical load is not placed on the subject and to the extent that the position of at least one pulse wave sensor does not deviate from the measurement site as long as the body motion of the subject is not excessively violent.

In a pulse wave measurement device of one embodiment, at least one pulse wave sensor is mounted on the belt. In a state in which the belt is mounted around the measurement site, the pressing member presses the at least one pulse wave sensor against the measurement site, for example, with a certain pressing force. In this state, each of the at least one pulse wave sensor detects a pulse wave in a facing portion of an artery passing through the measurement site. The body motion detection unit detects the presence or absence of body motion of the subject. When there is no body motion of the subject, the control unit sets a pressing force of the pressing member to a first pressing force to measure a pulse wave with the at least one pulse wave sensor. When there is body motion of the subject, the control unit sets a pressing force of the pressing member to a second pressing force lower than the first pressing force and higher than zero and interrupts measurement of a pulse wave. Thus, when there is body motion of the subject, it is possible to set a pressing force of the pressing member to the second pressing force to alleviate the physical burden on the subject. In addition, since the second pressing force is higher than zero, the position of at least one pulse wave sensor can be less likely to be deviated from the measurement site. As described above, controlling the pressing force on the measurement site by a novel control method in consideration of the body motion of the subject allows the convenience of the subject to be improved.

In the pulse wave measurement device of one embodiment, when measurement of a pulse wave is interrupted and then a state where there is body motion of the subject is transitioned to a state where there is no body motion of the subject, the control unit returns the pressing force of the pressing member to the first pressing force to resume measurement of a pulse wave.

In the pulse wave measurement device of the one embodiment, since the pressing force of the pressing member is set to a second pressing force higher than zero when the measurement of the pulse wave is interrupted, when pulse wave measurement is resumed, the pressing force of the pressing member can be returned to the first pressing force more quickly than when the pressing force of the pressing member is set to zero. Thus, the convenience of the subject can be improved.

In the pulse wave measurement device of one embodiment, when measurement of a pulse wave is interrupted and then a standby time having a predetermined length elapses, the control unit sets a pressing force of the pressing member to zero.

In the pulse wave measurement device of this one embodiment, it is possible to avoid useless pressing at the measurement site.

In the pulse wave measurement device of one embodiment, the pulse wave measurement device comprises a first pulse wave sensor and a second pulse wave sensor mounted on the belt in a state of being separated from each other in a width direction of the belt, each of the first pulse wave sensor and the second pulse wave sensor configured to detect a pulse wave in a facing portion of an artery passing through the measurement site.

In the pulse wave measurement device according to this embodiment, the first pressing force is, for example, set to a value at which the cross-correlation coefficient of the first and second pulse wave signals respectively output in time series by the first and second pulse wave sensors exceeds a predetermined threshold value. Here, the “cross-correlation coefficient” means the sample correlation coefficient (also referred to as Pearson's product-moment correlation coefficient). For example, when a data sequence {xᵢ} and a data sequence {yᵢ} including two sets of numerical values (where i=1, 2, . . . , n) are given, the cross-correlation coefficient r between the data sequence {xᵢ} and the data sequence {yᵢ} is defined by the equation (Eq. 4) illustrated in FIG. 16. In the equation (Eq. 4), x and y with overline respectively represent average values of x and y.

In the pulse wave measurement device of one embodiment, the first and second pulse wave sensors are mounted on the belt in a state of being separated from each other in the width direction of the belt. In a state in which the belt is mounted around the measurement site, the pressing member presses the first and second pulse wave sensors against the measurement site, for example, with a certain pressing force. In this state, each of the first and second pulse wave sensors detects a pulse wave in a facing portion of an artery passing through the measurement site. The body motion detection unit detects the presence or absence of body motion of the subject. When there is no body motion of the subject, the control unit sets a pressing force of the pressing member to a first pressing force to measure a pulse wave with the first and second pulse wave sensors. When there is no body motion of the subject, the control unit sets a pressing force of the pressing member to a first pressing force to measure a pulse wave with the first and second pulse wave sensors. Thus, when there is body motion of the subject, it is possible to set a pressing force of the pressing member to the second pressing force to alleviate the physical burden on the subject. In addition, since the second pressing force is higher than zero, the position of the first and second pulse wave sensors can be less likely to be deviated from the measurement site. As described above, controlling the pressing force on the measurement site by a novel control method in consideration of the body motion of the subject allows the convenience of the subject to be improved.

In the pulse wave measurement device of one embodiment, the pressing member includes an element configured to press the first pulse wave sensor and the second pulse wave sensor with an individual pressing force, and the control unit sets the first pressing force of the pressing member to individual values with respect to the first pulse wave sensor and the second pulse wave sensor.

In the pulse wave measurement device of this one embodiment, setting the first pressing force of the pressing member to individual values with respect to the first and second pulse wave sensors allows measurement accuracy of a pulse wave and blood pressure to be improved.

In another aspect, a blood pressure measurement device of the present disclosure comprises:

the pulse wave measurement device; and

a first blood pressure calculation unit configured to calculate blood pressure by using a predetermined correspondence equation between pulse transit time and blood pressure based on pulse transit time being a time difference between a first pulse wave signal and a second pulse wave signal respectively output in time series by the first pulse wave sensor and the second pulse wave sensor.

In the blood pressure measurement device of this one embodiment, the pulse wave measurement device acquires pulse transit time. The first blood pressure calculation unit calculates (estimates) the blood pressure based on the pulse transit time by using a predetermined correspondence equation between pulse transit time and blood pressure. Therefore, when the blood pressure of the subject is measured, controlling the pressing force on the measurement site by a novel control method in consideration of the body motion of the subject as described above allows the convenience of the subject to be improved.

In the blood pressure measurement device of one embodiment,

the pressing member is a fluid bag provided along the belt,

the blood pressure measurement device further comprises a main body provided integrally with the belt, and

wherein on the main body, the body motion detection unit, the control unit, and the first blood pressure calculation unit are mounted, and a pressure control unit configured to supply air to the fluid bag to control pressure, and a second blood pressure calculation unit configured to calculate blood pressure based on pressure in the fluid bag are mounted for blood pressure measurement by oscillometric method.

Herein, the main body being “integrally provided” with respect to the belt may mean that the belt and the main body are, for example, integrally molded, or instead of this, may mean that the belt and the main body may be separately formed, and the main body may be integrally attached to the belt via an engaging member (for example, a hinge or the like).

In the pulse wave measurement device of this one embodiment, the blood pressure measurement (estimation) based on pulse transit time and the blood pressure measurement by oscillometric method can be performed by an integrated device. Therefore, the convenience of the user is enhanced.

In another aspect, a pulse wave measurement method of the present disclosure is a pulse wave measurement method includes:

using
a belt to be mounted around a measurement site of a subject,
at least one pulse wave sensor mounted on the belt, the at least one pulse wave sensor configured to detect a pulse wave of an artery passing through the measurement site,
a pressing member mounted on the belt, the pressing member configured to vary a pressing force to press the at least one pulse wave sensor against the measurement site, and
a body motion detection unit configured to detect presence or absence of body motion of the subject,

to measure a pulse wave of the measurement site, the pulse wave measurement method comprising:

setting a pressing force of the pressing member to a first pressing force when there is no body motion of the subject to measure a pulse wave with the at least one pulse wave sensor; and

setting a pressing force of the pressing member to a second pressing force lower than the first pressing force and higher than zero when there is body motion of the subject and interrupting measurement of a pulse wave.

In the pulse wave measurement method of this one embodiment, it is possible to avoid useless pressing at the measurement site.

The above embodiments are illustrative, and various modifications can be made without departing from the scope of the present invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A pulse wave measurement device comprising:
a belt to be mounted around a measurement site of a subject;
at least one pulse wave sensor mounted on the belt, the at least one pulse wave sensor configured to detect a pulse wave of an artery passing through the measurement site;
a pressing member mounted on the belt, the pressing member configured to vary a pressing force to press the at least one pulse wave sensor against the measurement site;
a body motion detection unit configured to detect presence or absence of body motion of the subject; and
a control unit configured to set a pressing force of the pressing member to a first pressing force when there is no body motion of the subject to measure a pulse wave with the at least one pulse wave sensor, the control unit configured to set a pressing force of the pressing member to a second pressing force lower than the first pressing force and higher than zero when there is body motion of the subject and interrupt measurement of a pulse wave.

2. The pulse wave measurement device according to claim 1, wherein when measurement of a pulse wave is interrupted and then a state where there is body motion of the subject is transitioned to a state where there is no body motion of the subject, the control unit returns the pressing force of the pressing member to the first pressing force to resume measurement of a pulse wave.

3. The pulse wave measurement device according to claim 1, wherein when measurement of a pulse wave is interrupted and then a standby time having a predetermined length elapses, the control unit sets a pressing force of the pressing member to zero.

4. The pulse wave measurement device according to any one of claim 1, further comprising a first pulse wave sensor and a second pulse wave sensor mounted on the belt in a state of being separated from each other in a width direction of the belt, each of the first pulse wave sensor and the second pulse wave sensor configured to detect a pulse wave in a facing portion of an artery passing through the measurement site.

5. The pulse wave measurement device according to claim 4, wherein
the pressing member includes an element configured to press the first pulse wave sensor and the second pulse wave sensor with an individual pressing force, and
the control unit sets the first pressing force of the pressing member to individual values with respect to the first pulse wave sensor and the second pulse wave sensor.

6. A blood pressure measurement device comprising:
the pulse wave measurement device according to claim 4; and
a first blood pressure calculation unit configured to calculate blood pressure by using a predetermined correspondence equation between pulse transit time and blood pressure based on pulse transit time being a time difference between a first pulse wave signal and a second pulse wave signal respectively output in time series by the first pulse wave sensor and the second pulse wave sensor.

7. The blood pressure measurement device according to claim 6,
wherein the pressing member is a fluid bag provided along the belt,
the blood pressure measurement device further comprising a main body provided integrally with the belt, and
wherein on the main body, the body motion detection unit, the control unit, and the first blood pressure calculation unit are mounted, and a pressure control unit configured to supply air to the fluid bag to control pressure, and a second blood pressure calculation unit configured to calculate blood pressure based on pressure in the fluid bag are mounted for blood pressure measurement by oscillometric method.

8. A pulse wave measurement method including:
using
a belt to be mounted around a measurement site of a subject,
at least one pulse wave sensor mounted on the belt, the at least one pulse wave sensor configured to detect a pulse wave of an artery passing through the measurement site,
a pressing member mounted on the belt, the pressing member configured to vary a pressing force to press the at least one pulse wave sensor against the measurement site, and
a body motion detection unit configured to detect presence or absence of body motion of the subject,
to measure a pulse wave of the measurement site, the pulse wave measurement method comprising:
setting a pressing force of the pressing member to a first pressing force when there is no body motion of the subject to measure a pulse wave with the at least one pulse wave sensor; and
setting a pressing force of the pressing member to a second pressing force lower than the first pressing force and higher than zero when there is body motion of the subject and interrupting measurement of a pulse wave.
